(19) United States
(12) Patent Application Publication
(10) Pub. No.: US 2019/0223723 A1
(43) Pub. Date: Jul. 25, 2019

(54) SYSTEMS AND METHODS FOR MEASURING PATIENT VITAL SIGNS

(71) Applicant: Arvind Thiagarajan, Santa Clara, CA (US)

(72) Inventor: Arvind Thiagarajan, Santa Clara, CA (US)

(21) Appl. No.: 16/255,797

(22) Filed: Jan. 23, 2019

Related U.S. Application Data
(63) Continuation of application No. 15/478,189, filed on Apr. 3, 2017, now abandoned.
(60) Provisional application No. 62/319,770, filed on Apr. 7, 2016.

(51) Int. Cl. A61B 5/00; A61B 7/04; A61B 5/0408

(52) U.S. Cl. CPC A61B 5/0006; A61B 7/04; A61B 5/002; A61B 5/025; A61B 5/04085; A61B 5/0408; A61B 5/0093

(57) ABSTRACT

Systems and methods for electronically monitoring chest sounds and/or sensing electrical cardiac signals such as ECG signals are provided. In one embodiment, a hybrid stethdiographer has a sensing assembly with a chestpiece and a user interface. Stethdiographer also includes a conduit, a power source compartment, a pair of binaurals and a corresponding pair of earpieces. The user interface includes a record button, a mode selector and a display screen. The chestpiece includes a diaphragm and a plurality of electrical cardiac sensors.

### CROSS REFERENCE TO RELATED APPLICATIONS

This Continuation Application claims the benefit of U.S. application Ser. No. 15/478,189, filed on Apr. 3, 2017, of the same title, which application is a Non-Provisional application and claims the benefit of U.S. Provisional Application No. 62/319,770, filed on Apr. 7, 2016, which applications are incorporated herein in their entirety by this reference.

### BACKGROUND

The present invention relates to systems and methods for electronically monitoring chest sounds and/or ECG (electrocardiogram) of a subject.

Currently, electronic stethoscopes have emerged to overcome some of these limitations of acoustic stethoscopes. Most of these electronic stethoscopes are capable of amplifying and filtering the acoustic signals thereby substantially increasing their capabilities over the acoustic stethoscopes.

However, in addition to being able to monitor chest sounds, there is often a need for primary care providers and emergency services personnel to measure electrical cardiac signals, e.g., ECG signals, which are beyond the capability of these modern electronic stethoscopes.

It is therefore apparent that an urgent need exists for hybrid devices that are able to measure chest sounds and/or ECG signals. These improved hybrid devices have the selectable multi-purpose capability while substantially retaining the familiarity and resulting ease of use associated with stethoscopes in daily use by primary care providers and emergency services personnel.

### SUMMARY

To achieve the foregoing and in accordance with the present invention, systems and methods for electronically monitoring chest sounds and/or electrical cardiac signals such as ECG signals are provided.

In one embodiment, a hybrid stethdiographer has a sensing assembly with a chestpiece and a user interface. Stethdiographer also includes a conduit, a compartment for a power source, a pair of Y-splits, a pair of binaurals and a corresponding pair of earpieces.

In this embodiment, the user interface includes a record button, a mode selector, a display screen, a rewind button, a pause button, a fast forward button and a power activator and/or indicator. The chestpiece includes a diaphragm and a housing accommodating a plurality of electro-cardio sensors.

In some embodiments, stethdiographer also includes one or more ECG sensors on the top surface of the sensing assembly so as to be able to record signals from the subject's fingertips, signals that are traditionally measured using the subject's limbs.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIGS. 1A and 1B are top and bottom perspective views, respectively, illustrating one embodiment of a hybrid stethdiographer in accordance with the present invention;

FIGS. 2 and 3 illustrate an exemplary operating environment for the hybrid stethdiographer of FIGS. 1A-1B;

FIGS. 4A and 4B depict exemplary screenshots illustrating the operation of the hybrid stethdiographer of FIGS. 1A-1B;

FIG. 5 is a top perspective view illustrating another embodiment of a hybrid stethdiographer in accordance with the present invention;

FIGS. 6A-6B and 6C depict exploded and assembled perspective views illustrating assembly of one embodiment of a chestpiece for the hybrid stethdiographer of FIGS. 1A-1B;

FIGS. 7A-7B and 7C depict exploded and assembled perspective views illustrating assembly of another embodiment of a chestpiece for the hybrid stethdiographer of FIGS. 1A-1B;

FIGS. 8A and 8B depict cross-sectional perspective views illustrating one embodiment of a cardiac sensor for the hybrid stethdiographer of FIGS. 1A-1B; and

FIG. 9 depicts cross-sectional perspective views illustrating an alternative means for securing a cardiac sensor to the hybrid stethdiographer of FIGS. 1A-1B.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

Aspects, features and advantages of exemplary embodiments of the present invention will become better understood with regard to the following description in connection with the accompanying drawing(s). It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto. Hence, use of absolute and/or sequential terms, such as, for example, “always,” “only,” “will,” “will not,” “shall,” “shall not,” “must,” “must not,” “first,” “initially,” “next,” “subsequently,” “before,” “after,” “lastly,” and “finally,” are not meant to limit the scope of the present invention as the embodiments disclosed herein are merely exemplary.

The present invention relates to systems and methods for electronically monitoring chest sounds and/or electrical cardiac signals such as ECG signals thereby alleviating the need for multiple discrete medical devices such as having both stethoscopes in addition to electrocardiogram machines.

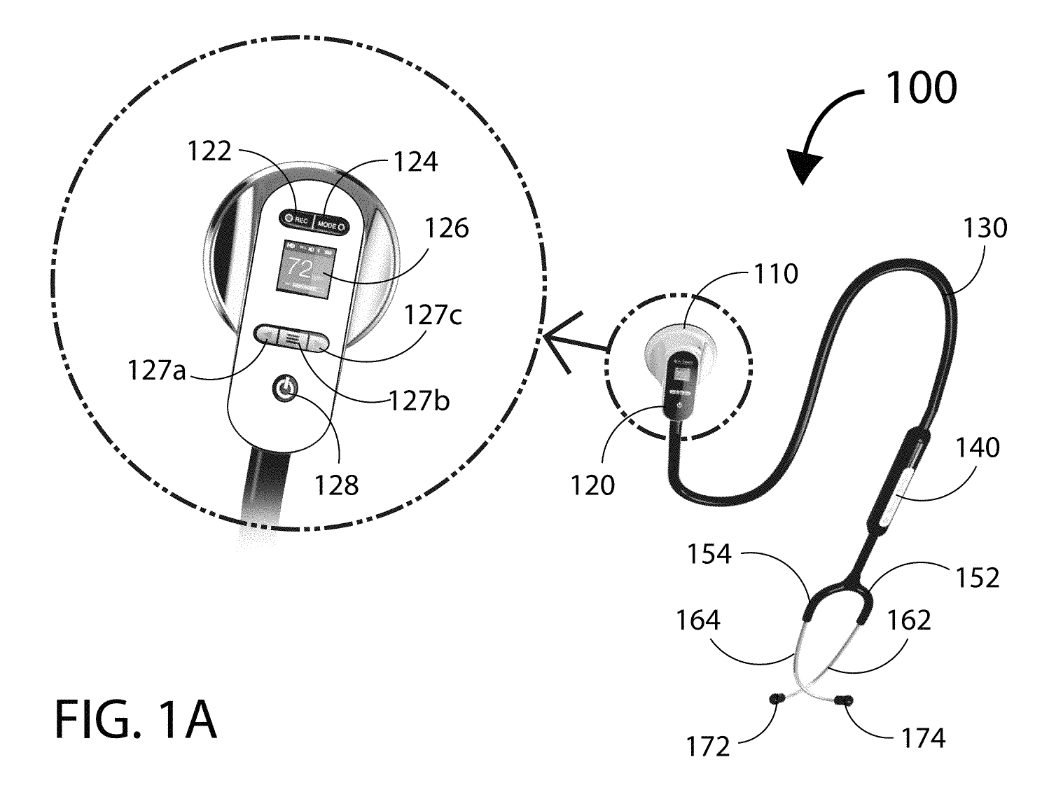

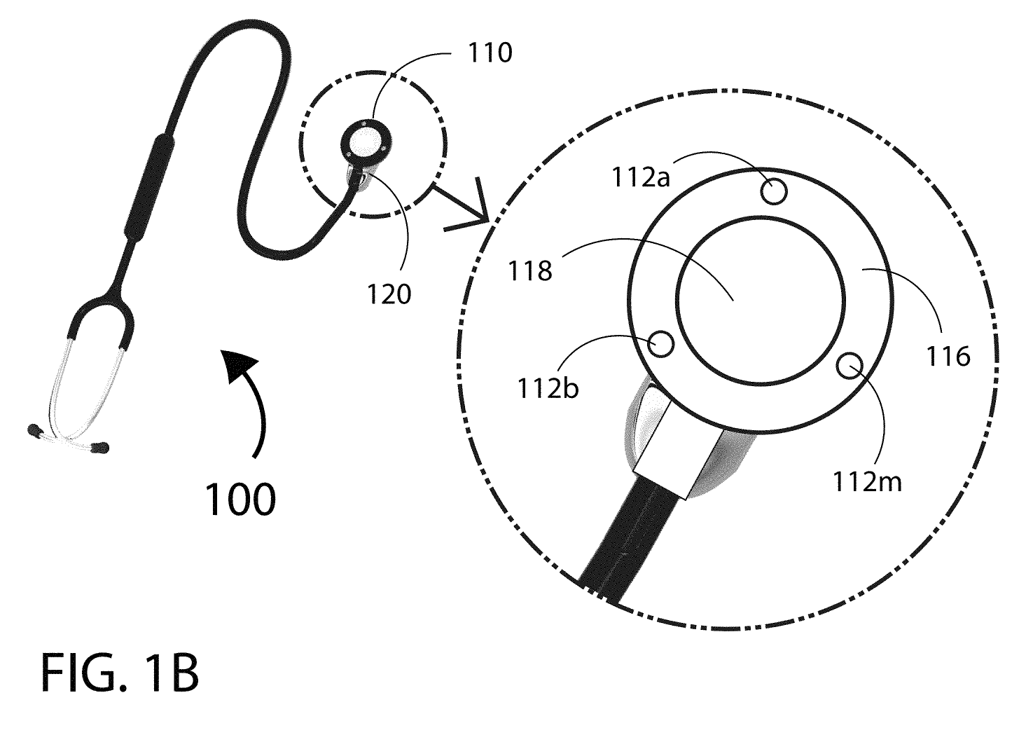

To facilitate discussion, FIGS. 1A and 1B are top and bottom perspective views, respectively, illustrating one embodiment of a hybrid stethdiographer 100 in accordance with the present invention.

In this embodiment stethdiographer 100 includes a sensing assembly with a chestpiece 110 and a user interface 120. Stethdiographer 100 also includes a conduit 130, a compartment 140, a pair of Y-splits 152, 154, a pair of binaurals 162, 164 and a corresponding pair of earpieces 172, 174.

FIG. 1A also depicts an enlarged top view of the user interface 120 having a record button 122, a mode selector 124, a display screen 126, a rewind button 127a, a pause button 127b, a fast forward button 127c and a power activator and/or indicator 128. FIG. 1B depicts an enlarged bottom view of chestpiece 110 having a diaphragm 118 and a housing 116 accommodating a plurality of electro-cardio sensors 112a, 112b . . . 112m configured to sense a subset of V1-V6 heart signals (see Appendix A).

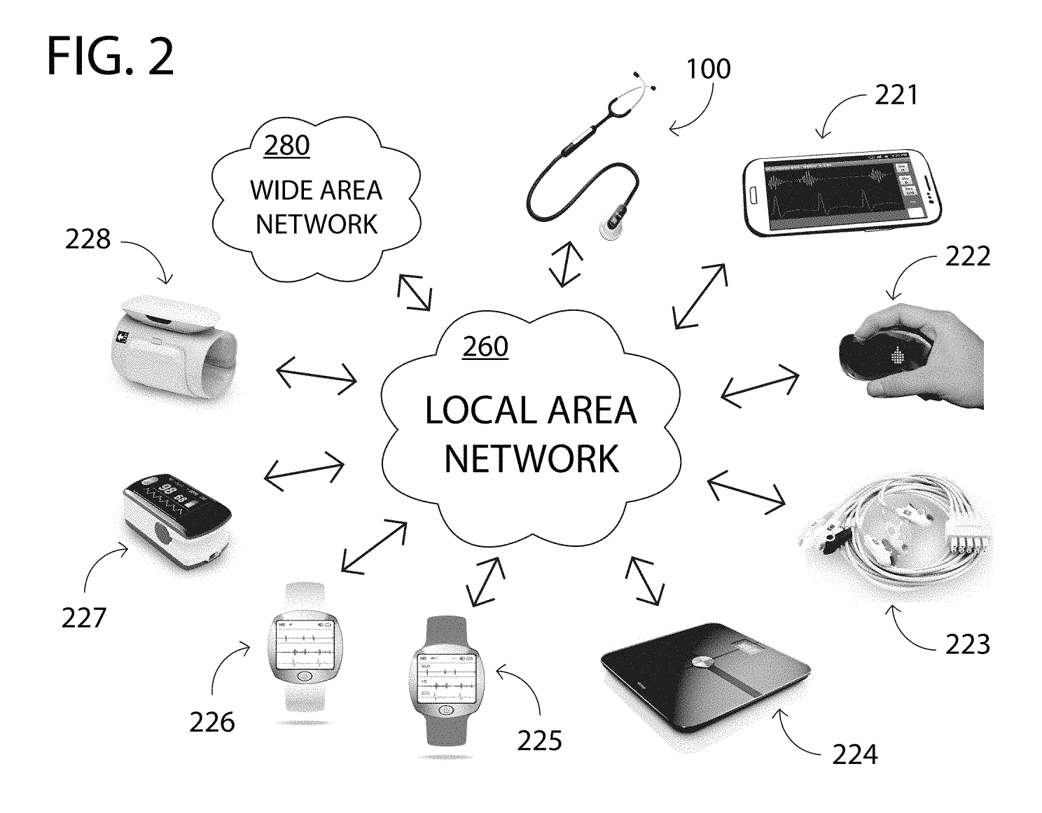

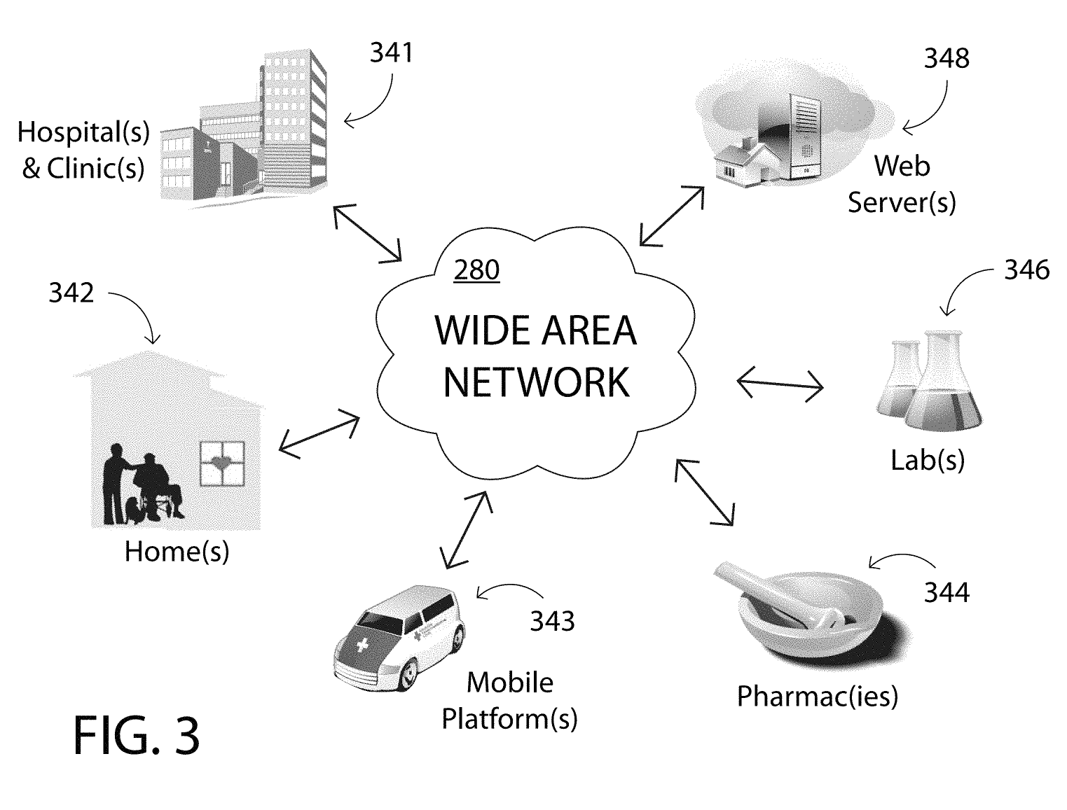

Together FIGS. 2 and 3 illustrate an exemplary operating environment for stethdiographer 100. For example, a medical facility, such as a primary care clinic 341 can support a wide variety of devices 100, 221, 222, 223, 224, 225, 226, 227, 228 communicating with each other via a local area network 260. These devices 100, 221, 222, 223, 224, 225, 226, 227, 228 can in turn communicate, via WAN 280, with one or more devices of other device clusters (not shown) inside other localities such as hospitals 341, long term care homes 342, ambulances 343, pharmacies 344, laboratories 346 and remote servers 348 associated with, for example, health insurance companies.

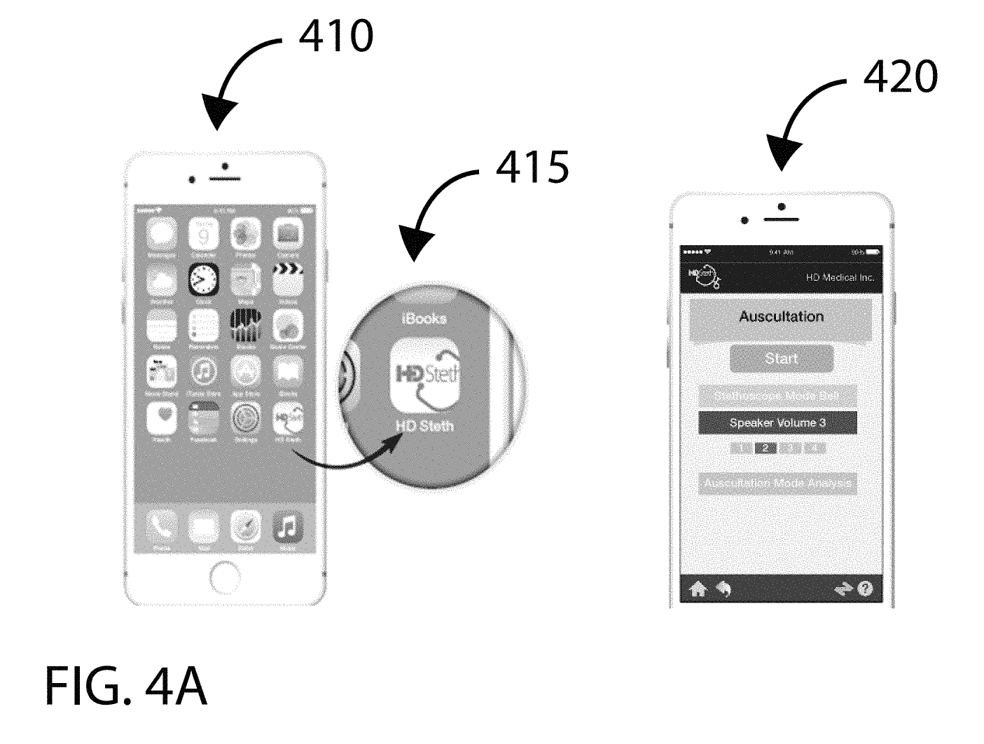

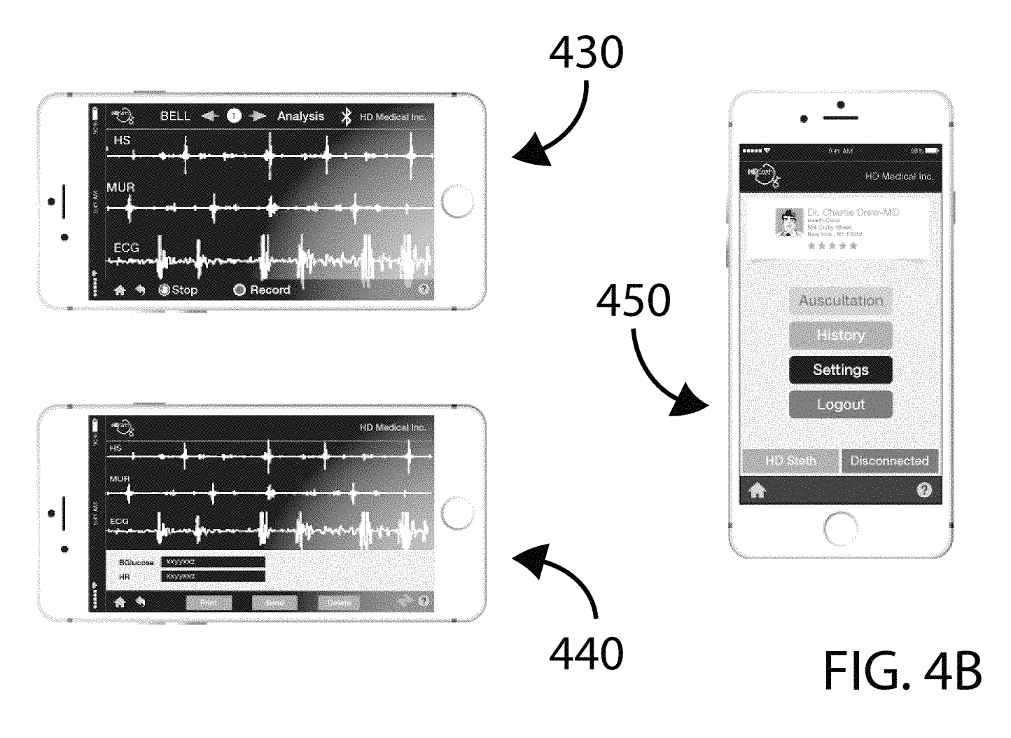

FIGS. 4A and 4B depict exemplary screenshots 410, 415, 420 and exemplary screenshots 430, 440, 450, respectively, illustrating the operation of stethdiographer 100.

Referring back to FIGS. 1A and 1B, stethdiographer 100 is configured to operate in one or more of the following Modes.

1) Stethoscope Auscultation Modes:
a) Bell
b) DIA
c) LUNG/WIDE

2) Stethoscope Functional Modes:
a) Electronic Stethoscope Mode
b) Clinical Mode
c) Analysis Mode
d) Tele-Med Mode
e) Tele-Med Live Mode

3) Record, Save, Replay and Transfer 10 on device stored Heart Sounds
4) Data transfer using Bluetooth and/or USB cable
5) Dedicated Record Start and Stop Button
6) Dedicated Auscultation Mode selection button
7) Heart Rate Display

A) Electronic Stethoscope Mode (Standard Activation)

In this mode, stethdiographer 100 can function as a conventional electronic stethoscope, i.e., a user can auscultate in BELL/DIA/LUNG mode:
i) Record Heart Sound and ECG data
ii) Save Heart Sounds and ECG Data
iii) Replay Heart Sounds in desired auscultation mode

Transfer the recorded Heart Sounds and ECG Data by switching on the Bluetooth. In this mode, a HD APP (an exemplary software application) executing on external devices, for example, one or more of mobile devices 221, 224, 225, 226, is not yet synced with stethdiographer 100 and hence the HD APP features may not active.

B) Clinical Mode (Standard Activation)

In Clinical mode, the functions of Basic Stethoscope Mode are available on stethdiographer 100. Additionally, user can use the HD APP functions when stethdiographer 100 is synced with the HD APP. Accordingly, the user can auscultate in BELL/DIA/LUNG mode:
i) Record Heart Sound and ECG data
ii) Save Heart Sounds and ECG Data
iii) Replay Heart Sounds in desired auscultation mode
iv) Transfer recorded/saved Heart Sounds and ECG data
v) Real time PCG and ECG wave form on HD APP display screen
vi) Replay recorder and saved Heart Sounds and ECG Data on HD APP
vii) Hear saved Heart Sounds on HD APP using stethdiographer 100.
viii) Hear saved Heart Sounds on HD APP using external speakers (Headphones, Mobile/PC/Tablet speakers)

C) Analysis Mode (Standard Activation)

In Analysis mode, the functions of Clinical Mode are available via stethdiographer 100. Additionally, the user can hear real time Heart Sounds on stethdiographer 100. With stethdiographer 100, the user can also auscultate in BELL/DIA/LUNG mode:
i) Record Heart Sound and ECG data
ii) Save Heart Sounds and ECG Data
iii) Replay Heart Sounds in desired auscultation mode
iv) Transfer recorded/saved Heart Sounds and ECG data
v) Real time PCG and ECG waver form on HD APP display screen with color indicators for Heart Sound and ECG anomalies.
vi) Real Time Heart Sounds can be heard using HD APP device using external speakers (Headphones, Mobile/PC/Tablet speakers)
vii) Replay recorder and saved Heart Sounds and ECG Data on HD APP
viii) Hear saved Heart Sounds on HD APP using stethdiographer 100
ix) Hear saved Heart Sounds on HD APP using external speakers (Headphones, Mobile/PC/Tablet speakers)

D) Tele-Med LIVE Mode (Remote Activation)

Referring also to FIGS. 2 and 3, in Tele-Med mode, the functions of Analysis Mode can also be available locally using stethdiographer 100. Additionally, the user can push the Heart Sounds and ECG data to a HD APP device via LAN 260 and/or WAN 280, thereby enabling a remote user to access the data and can replay the recorded Heart Sound and ECG data using HD APP on the remote device. User can also auscultate in BELL/DIA/LUNG mode:
i) Record Heart Sound and ECG data
ii) Save Heart Sounds and ECG Data
iii) Replay Heart Sounds in desired auscultation mode
iv) Transfer recorded/saved Heart Sounds and ECG data
v) Real time PCG and ECG waver form on HD APP display screen with color indicators for Heart Sound and ECG anomalies
vi) Real Time Heart Sounds can be heard using HD APP device using external speakers (Headphones, Mobile/PC/Tablet speakers)
vii) Replay recorder and saved Heart Sounds and ECG Data on HD APP
viii) Push the recorder/saved Hear Sound and ECG Data via cloud to remote user
ix) Remote user can hear received Heart Sounds on HD APP using stethdiographer 100
x) Remote user can hear saved Heart Sounds on HD APP using external speakers (Headphones, Mobile/PC/Tablet speakers)

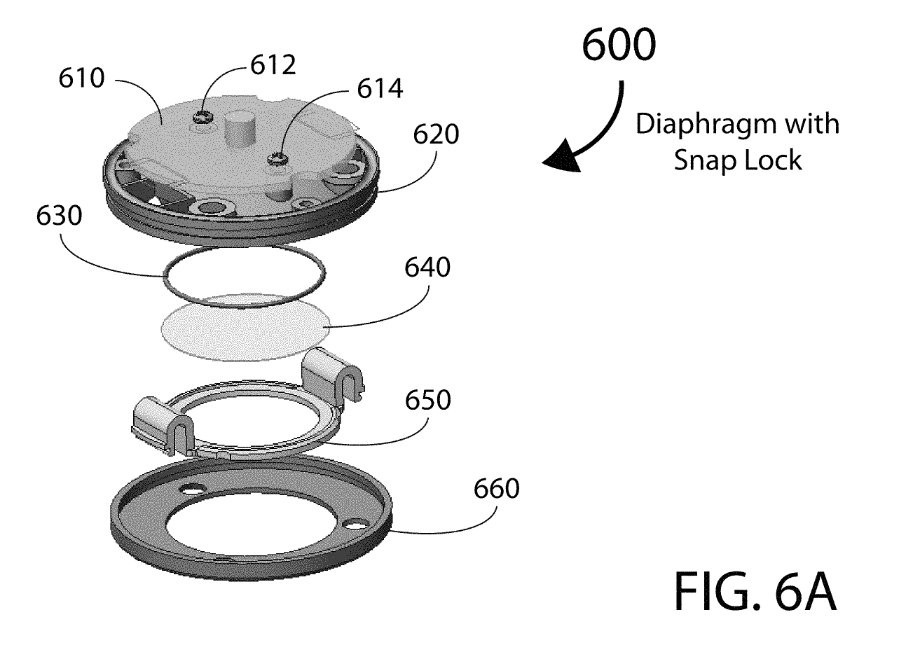

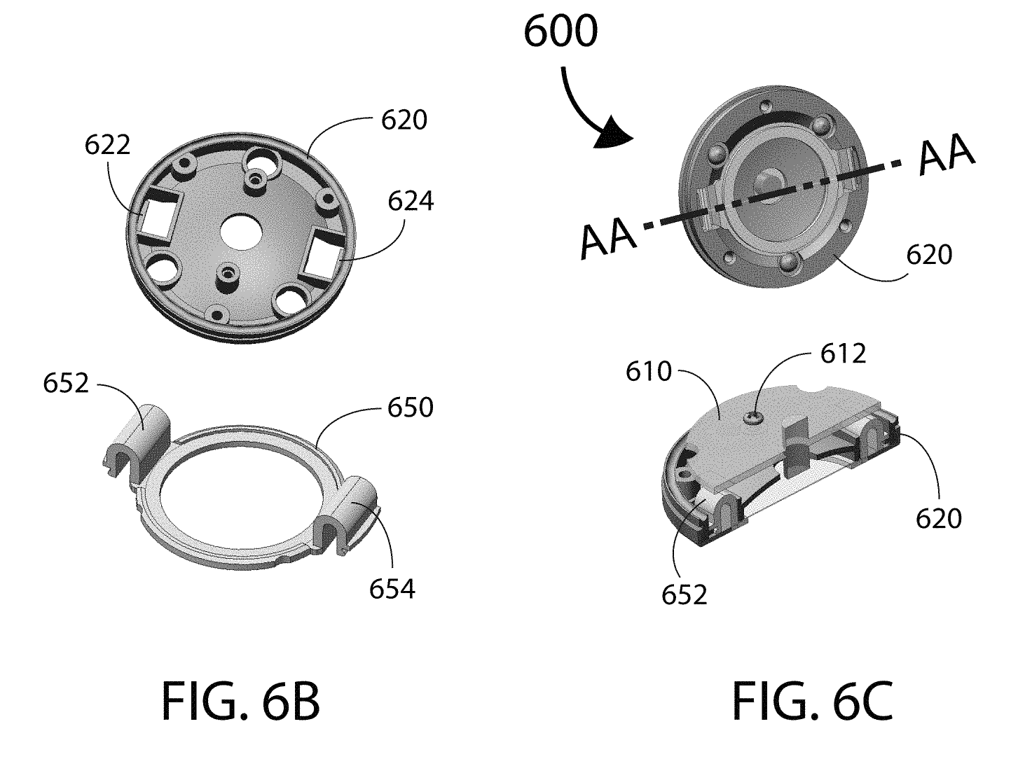

In some embodiments, as shown in FIGS. 6A and 6B, exploded perspective views illustrating an exemplary chestpiece subassembly 600 for hybrid stethdiographer 100, sub-assembly 600 includes a printed circuit board (PCB) 610, a PCB holder 620, an O-ring 630, a diaphragm 640, a diaphragm holder 650 with snap locks 652, 654 and a chestpiece cover 660. FIG. 6C includes a cross-sectional view AA-AA of sub-assembly 600 depicting how diaphragm holder 650 can be snapped into recesses 622, 624 of PCB holder 620. Cover 660 may provide concealment for screws 612, 614 (not shown) configured to secure the above described components of sub-assembly 600 to each other. Cover 660 can be made from a suitable material such as rubber or foam.

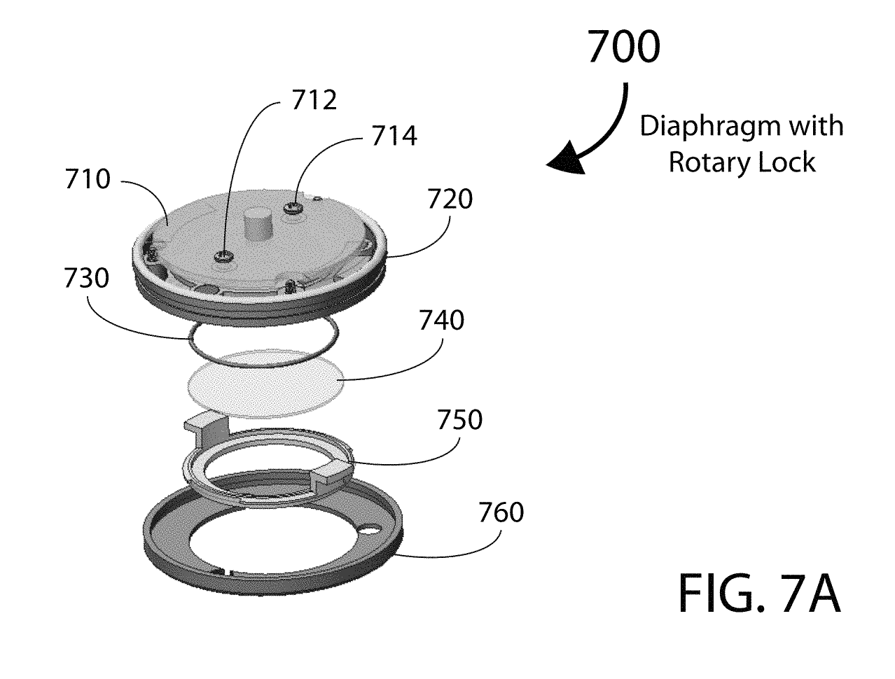

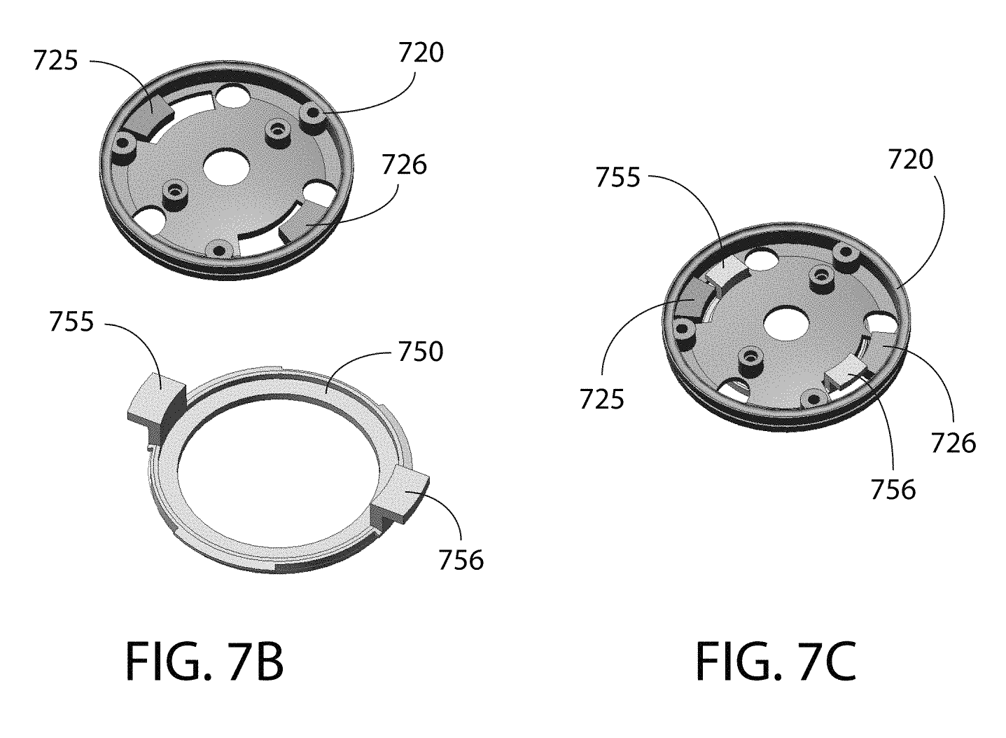

FIGS. 7A-7B and 7C depict exploded and assembled perspective views illustrating another embodiment of a chestpiece subassembly 700 for hybrid stethdiographer 100. Sub-assembly 700 includes a PCB 710, a PCB holder 720, an O-ring 730, a diaphragm 740, a diaphragm holder 750 and a chestpiece cover 760. In this embodiment, diaphragm holder 750 includes a pair of locking stubs 755, 756 configured to be rotatably secured to a corresponding pair of ramps 725, 726 of PCB holder 720, thereby increasing the engagement of O-ring 730 and diaphragm 740, compressed and sandwiched between diaphragm holder 750 and PCB holder 720.

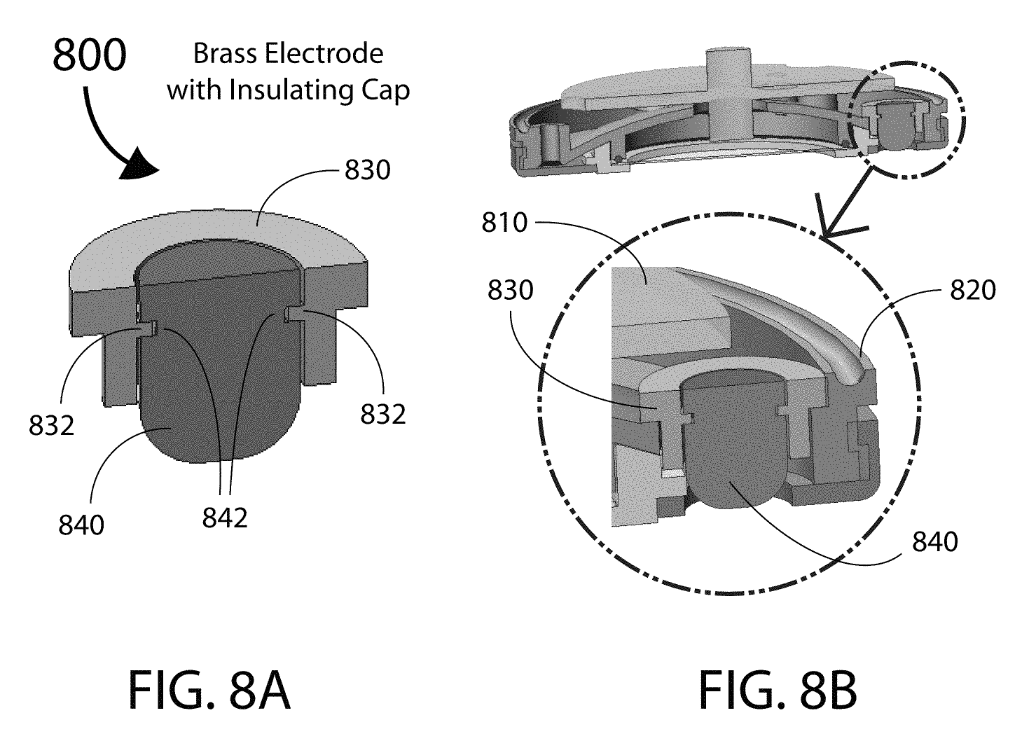

Referring now to FIGS. 8A and 8B, cross-sectional perspective views illustrating one embodiment of a cardiac sensor 800 for hybrid stethdiographer 100. Cardiac sensor 800 includes an electrode 840 and an insulating cap 830. In this embodiment, electrode 840 has a groove 842 configured to mate with a corresponding ridge 832 of cap 830 during assembly. Once assembled, sensor 800 is configured to be seated securely in PCB holder 820 from downward pressure exerted by printed circuit board (PCB) 810, as shown in FIG. 8B. Electrode 840 can be made from a suitable conductor such as brass. Cap 830 can be made from a suitable insulator such as plastic.

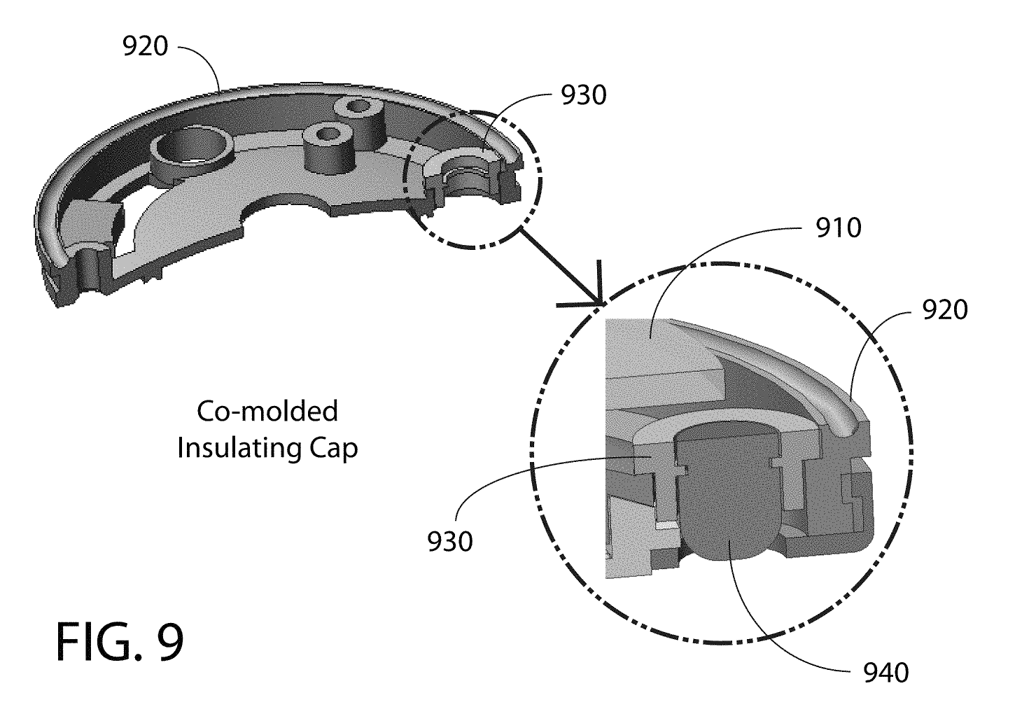

FIG. 9 includes cross-sectional perspective views depicting an alternative cardiac sensor subassembly for hybrid stethdiographer 100. In this embodiment, hybrid stethdiographer 100 includes a PCB holder 920 with at least one co-molded insulating cap 930 configured to house a corresponding electrode 940. During assembly, electrode 940 is inserted into co-molded cap 930 and seated securely in PCB holder 920 from downward pressure exerted by printed circuit board (PCB) 910.

In some embodiments, in addition to having an internal sound transducer such as a microphone (not shown) operatively coupled to the diaphragm (e.g., diaphragm 118, 640 or 740), hybrid stethdiographers (e.g., stethdiographer 100) may also include an external sound transducer (not shown) configured to sense ambient sounds thereby enabling these hybrid stethdiographers to provide ambient noise cancellation. In addition, the existence of a secondary external sound transducer such as a microphone may also enable these hybrid stethdiographers to provide hands-free dictation, transcription and/or real-time translation capabilit(ies) for the user.

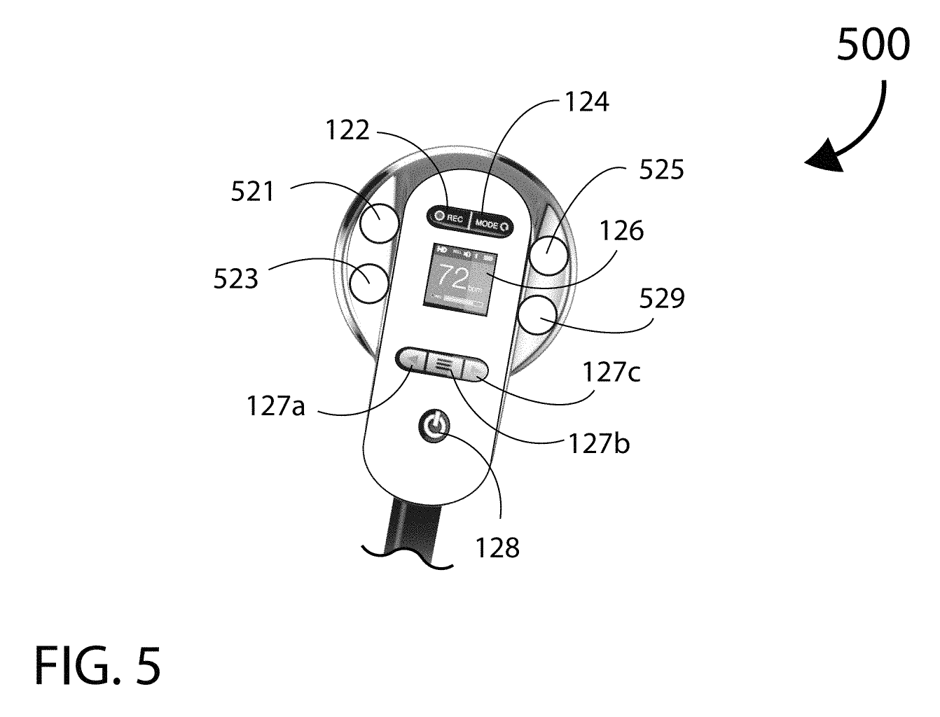

Many modifications and additions are to the above described embodiment of hybrid stethdiographer 100 are possible. For example, as shown in FIG. 5, stethdiographer 500 also include additional sensors 521, 523, 525, 529 configured to measure a subset of RL, RA, LL and LA signals from the subject's fingertips (see Appendix A). In addition, one of these sensors 521, 523, 525, 529 may be a fingerprint sensor to facilitate user and/or subject identification.

While this invention has been described in terms of several embodiments, there are alterations, modifications, permutations, and substitute equivalents, which fall within the scope of this invention. Although sub-section titles have been provided to aid in the description of the invention, these titles are merely illustrative and are not intended to limit the scope of the present invention.

It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A portable hybrid stethdiographer configured to measure chest sounds and cardiac signals, the stethdiographer comprising:
a sensing assembly including a chestpiece and a user interface, wherein the chestpiece includes a diaphragm configured to sense chest sounds from a subject, and wherein the chestpiece also includes a plurality of cardiac sensors configured to sense cardiac signals from the subject;
a conduit configured to transfer the chest sounds from the sensing assembly;
a compartment;
a pair of Y-splits configured to split the chest sounds from the conduit;
a pair of binaurals configured to channel the chest sounds from the Y-splits; and
a pair of earpieces configured to provide chest sounds from the binaurals to a user.

2. The hybrid stethdiographer of claim 1 wherein the compartment is configured to house a power source.

3. The hybrid stethdiographer of claim 1 wherein the cardiac signals are ECG (electrocardiogram) signals.

4. The hybrid stethdiographer of claim 1 wherein the user interface includes a record button, a mode selector, a display screen, a rewind button, a pause button, a fast forward button and a power activator or indicator.

5. The hybrid stethdiographer of claim 4 wherein the display screen is configured to display at least one of the chest sounds and the cardiac signals.

6. The hybrid stethdiographer of claim 1 further comprising a first and a second top cardiac sensors configured to sense additional cardiac signals from at least two fingertips of the subject.

7. The hybrid stethdiographer of claim 6 wherein the additional cardiac signals are ECG signals.

8. The hybrid stethdiographer of claim 6 wherein the display screen is configured to display at least one of the additional cardiac signals.

9. The hybrid stethdiographer of claim 1 further comprising a fingerprint sensor.

10. The hybrid stethdiographer of claim 1 wherein each of the plurality of cardiac sensors includes an insulating cap and an electrode.

11. The hybrid stethdiographer of claim 1 further comprising an internal sound transducer operatively coupled to the diaphragm.

12. The hybrid stethdiographer of claim 1 further comprising an external sound transducer configured to sense ambient sounds thereby providing ambient noise cancellation.

13. The hybrid stethdiographer of claim 12 wherein the external sound transducer is further configured to provide at least one of hands-free dictation, transcription and real-time translation.
